(19) United States
(12) Patent Application Publication
(10) Pub. No.: US 2022/0202358 A1
(43) Pub. Date: Jun. 30, 2022

(54) ELECTRONIC DEVICE AND METHOD FOR DETECTING APNEA

(71) Applicant: Industrial Technology Research Institute, Hsinchu (TW)

(72) Inventors: Ting-Wu Ho, Hsinchu City (TW); Feng-Jie Tsai, Tainan City (TW); Chia-Lung Liu, Hsinchu County (TW)

(73) Assignee: Industrial Technology Research Institute, Hsinchu (TW)

(21) Appl. No.: 17/136,021

(22) Filed: Dec. 29, 2020

(51) Int. Cl. A61B 5/00

(52) U.S. Cl. CPC A61B 5/4818; A61B 5/0004; A61B 5/6898; A61B 5/002; A61B 5/7225

(57) ABSTRACT

An electronic device and a method for detecting apnea are provided. The method includes: transmitting a wireless signal to a human subject, receiving an echo corresponding to the wireless signal, and obtaining channel state information (CSI) from the echo; generating a detection result according to the CSI; outputting the detection result. Here, the detection result indicates whether an apnea event has occurred on the human subject.

### TECHNICAL FIELD

The technical field relates to an electronic device and a method for detecting apnea.

### BACKGROUND

According to statistics, many patients around the world suffer from apnea, but only some of them has received treatment. Apnea may affect the health condition and the quality of life of the patients. For instance, the patients with apnea are prone to snoring during sleep, which may disturb the sleep of their partners. When an apnea event occurs, the airflow in the patient's respiratory tract may completely stop, resulting in a decrease in the patient's blood oxygen concentration. Long-term reduction in the blood oxygen concentration may cause the Alzheimer's disease.

In order to check whether a person suffers from apnea, the person must spend the night in a sleep center of the hospital for breathing monitor. If the breath of the person stops for more than ten seconds, it may be determined that the person suffers from apnea. However, it is difficult for ordinary people to afford expensive sleep centers, and it takes a lot of time to visit the sleep center for examination.

In response to the above, some devices on the market allow users to check apnea by themselves. However, these devices require contact with the users, which reduces the users' willingness to use the devices or affects the quality of the users' sleep. In addition, the devices cannot reduce the frequency of the apnea events.

### SUMMARY

The disclosure provides an electronic device and a method for detecting apnea, so as to detect whether an apnea event has occurred on a human subject in a non-contact manner.

In an embodiment of the disclosure, an electronic device for detecting apnea includes a processor, a storage medium, and a transceiver. The storage medium stores a plurality of modules. The processor is coupled to the storage medium and the transceiver and accesses and executes the modules. Here, the modules include a data collection module, a detection module, and an output module. The data collection module transmits a wireless signal to a human subject through the transceiver, receives an echo corresponding to the wireless signal through the transceiver, and obtains channel state information (CSI) from the echo. The detection module generates a detection result according to the CSI, wherein the detection result indicates whether an apnea event has occurred on the human subject. The output module outputs the detection result through the transceiver.

In an embodiment of the disclosure, the electronic device further includes an air cushion and an air pump. The air pump is connected to the air cushion and communicates with the transceiver. Here, the modules further include an air cushion control module. The air cushion control module inflates the air cushion by configuring the air pump through the transceiver in response to the apnea event.

In an embodiment of the disclosure, the detection module is further configured to: calculate a first-order differential signal corresponding to the CSI and detect the apnea event based on the first-order differential signal.

In an embodiment of the disclosure, the detection module is further configured to: generate a filtering signal corresponding to the CSI according to a filtering algorithm and differentiate the filtering signal to generate the first-order differential signal.

In an embodiment of the disclosure, the filtering algorithm is associated with at least one of the following: moving average filtering, high pass filtering, low pass filtering, and bandpass filtering.

In an embodiment of the disclosure, the detection module is further configured to: generate a plurality of frequency responses corresponding to the first-order differential signal, wherein the frequency responses respectively correspond to a plurality of time points; obtain a plurality of global maximum values corresponding to the frequency responses; generate a global maximum value curve corresponding to a first reference time slot according to the global maximum values, wherein the first reference time slot includes the time points; detect the apnea event according to the global maximum value curve.

In an embodiment of the disclosure, the detection module is further configured to: extract a second reference time slot from the first reference time slot according to a threshold, wherein the second reference time slot corresponds to a subset of the global maximum values, and each of the global maximum values in the subset is less than the threshold; determine that the apnea event is detected in response to the second reference time slot being greater than an apnea time slot.

In an embodiment of the disclosure, the detection module is further configured to: arrange the global maximum values from small to large to generate a global maximum value sequence and select an N^{th} global maximum value from the global maximum value sequence as the threshold, wherein N is the product of a ratio of the apnea time slot to the first reference time slot and the quantity of the global maximum values.

In an embodiment of the disclosure, the detection module is further configured to: generate an upper bound and a lower bound according to a median of the first-order differential signal; determine a data point larger than the upper bound or smaller than the lower bound in the first-order differential signal as an outlier, correct the outlier to generate a corrected first-order differential signal, and generate the frequency responses according to the corrected first-order differential signal.

In an embodiment of the disclosure, the detection module corrects the outlier according to one of the following steps: replacing the outlier with median; performing an interpolation calculation on a first data point earlier than the data point and a second data point later than the data point to correct the outlier, wherein the first data point and second data point are included in the first-order differential signal.

In an embodiment of the disclosure, a method for detecting apnea includes following steps. A wireless signal is transmitted to a human subject, an echo corresponding to the wireless signal is received, and CSI is obtained from the echo. According to the CSI, a detection result is generated and is output. Here, the detection result indicates whether an apnea event has occurred on the human subject.

In an embodiment of the disclosure, the method further includes: configuring an air pump to inflate an air cushion in response to the apnea event.

In an embodiment of the disclosure, the step of generating the detection result according to the CSI includes: calculating a first-order differential signal corresponding to the CSI and detecting the apnea event according to the first-order differential signal.

In an embodiment of the disclosure, the step of calculating the first-order differential signal corresponding to the CSI includes: generating a filtering signal corresponding to the CSI according to a filtering algorithm and differentiating the filtering signal to generate a first-order differential signal.

In an embodiment of the disclosure, the filtering algorithm is associated with at least one of the following: moving average filtering, high pass filtering, low pass filtering, and bandpass filtering.

In an embodiment of the disclosure, the step of detecting the apnea event according to the first-order differential signal includes: generating a plurality of frequency responses corresponding to the first-order differential signal, wherein the frequency responses respectively correspond to a plurality of time points; obtaining a plurality of global maximum values corresponding to the frequency responses; generating a global maximum value curve corresponding to a first reference time slot according to the global maximum values, wherein the first reference time slot includes the time points; detecting the apnea event according to the global maximum value curve.

In an embodiment of the disclosure, the step of detecting the apnea event according to the global maximum value curve includes: extracting a second reference time slot from the first reference time slot according to a threshold, wherein the second reference time slot corresponds to a subset of the global maximum values, and each of the global maximum values in the subset is less than the threshold; determining that the apnea event is detected in response to the second reference time slot being greater than an apnea time slot.

In an embodiment of the disclosure, the step of detecting the apnea event according to the global maximum value curve further includes: arranging the global maximum values from small to large to generate a global maximum value sequence and selecting an N^{th} global maximum value from the global maximum value sequence as the threshold, wherein N is the product of a ratio of the apnea time slot to the first reference time slot and the quantity of the global maximum values.

In an embodiment of the disclosure, the step of generating the frequency responses corresponding to the first-order differential signal includes: generating an upper bound and a lower bound according to a median of the first-order differential signal, determining a data point larger than the upper bound or smaller than the lower bound in the first-order differential signal as an outlier, and correcting the outlier to generate a corrected first-order differential signal and generating the frequency responses according to the corrected first-order differential signal.

In an embodiment of the disclosure, the step of correcting the outlier to generate the corrected first-order differential signal includes one of following steps: replacing the outlier with the median and performing an interpolation calculation on a first data point earlier than the data point and a second data point later than the data point to update the outlier, wherein the first data point and the second data point are included in the first-order differential signal.

In view of the above, the electronic apparatus provided in one or more embodiments of the disclosure may be applied to detect the CSI without contacting the human subject and may still determine whether the apnea event has occurred on the human subject. In addition, the electronic apparatus provided in one or more embodiments of the disclosure may also determine whether the air cushion is applied to move the head of the human subject, so as to prevent the occurrence of respiratory arrest events.

To make the disclosure more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram of an electronic device for detecting apnea according to an embodiment of the disclosure.

FIG. 2A is a schematic diagram illustrating channel state information (CSI) according to an embodiment of the disclosure.

FIG. 2B is a schematic diagram of a first-order differential signal according to an embodiment of the disclosure.

FIG. 2C is a schematic diagram of a filtering signal according to an embodiment of the disclosure.

FIG. 2D is a schematic diagram of a corrected first-order differential signal according to an embodiment of the disclosure.

FIG. 3 is a schematic diagram of obtaining global maximum values of frequency responses according to an embodiment of the disclosure.

FIG. 4 is a schematic diagram of determining an occurrence of an apnea event based on the global maximum values according to an embodiment of the disclosure.

FIG. 5 is a schematic diagram of moving the head of a human subject through an air cushion according to an embodiment of the disclosure.

FIG. 6 is a flowchart of a method for detecting apnea according to an embodiment of the disclosure.

### DETAILED DESCRIPTION OF DISCLOSED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

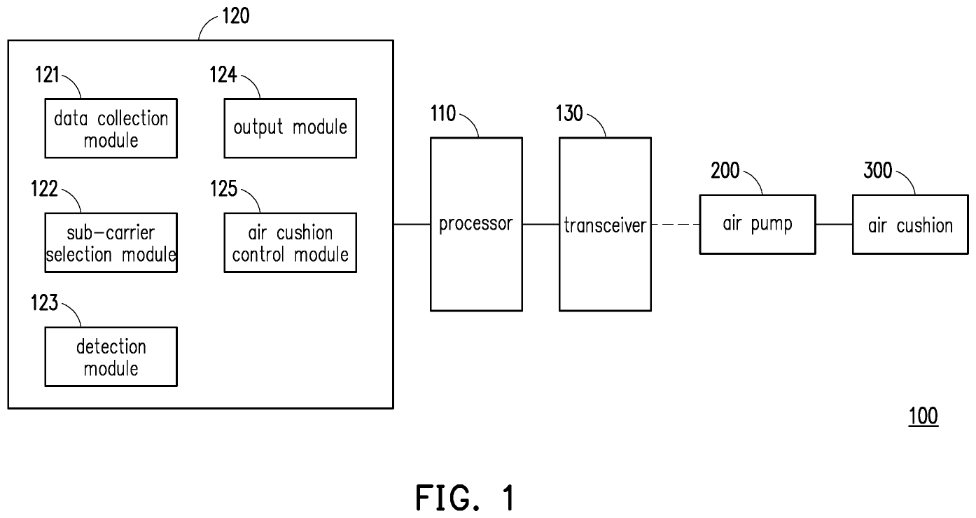

FIG. 1 is a schematic diagram of an electronic device 100 for detecting apnea according to an embodiment of the disclosure. The electronic device 100 may include a processor 110, a storage medium 120, and a transceiver 130. In an embodiment, the electronic device 100 may further include an air pump 200 and an air cushion 300.

The processor 110 is, for instance, a central processing unit (CPU) or any other programmable general-purpose or special-purpose micro control unit (MCU), microprocessor, digital signal processor (DSP), programmable controller, application specific integrated circuit (ASIC), graphics processing unit (GPU), image signal processor (ISP), image processing unit (IPU), arithmetic logic unit (ALU), complex programmable logic device (CPLD), field programmable gate array (FPGA), any other similar component, or a combination of the above components. The processor 110 may be coupled to the storage medium 120 and the transceiver 130 and may access and execute a plurality of modules and various application programs stored in the storage medium 120.

The storage medium 120 is, for instance, any type of fixed or portable random access memory (RAM), read only memory (ROM), flash memory), hard disk drive (HDD), solid state drive (SSD), any other similar component, or a combination of the above components, and the storage medium 120 may be configured to store the modules or various application programs that may be executed by the processor 110. In the embodiment, the storage medium 120 may store the modules including a data collection module 121, a sub-carrier selection module 122, a detection module 123, an output module 124, and an air cushion control module 125, and the functions of these modules are described hereinafter.

The transceiver 130 transmits and receives signals in a wireless or wired manner. The transceiver 130 may also perform operations including low noise amplification, impedance matching, frequency mixing, upward or downward frequency conversion, filtering, amplification, and the like. The processor 110 may communicate with the air pump 200 through the transceiver 130.

The air pump 200 is coupled to the air cushion 300. The processor 110 may be equipped with the air pump 200 to inflate or deflate the air cushion 300.

In order to detect whether an apnea event occurs in a human subject, the data collection module 121 may transmit a wireless signal to the human subject through the transceiver 130 and receive an echo corresponding to the wireless signal through the transceiver 130. The wireless signal is, for instance, any kind of radio frequency signal (e.g., a Wi-Fi signal). In an embodiment, the sub-carrier selection module 122 may select a specific sub-carrier. The data collection module 121 may transmit the wireless signal or receive the echo of the wireless signal from the selected sub-carrier through the transceiver 130. For instance, if the wireless signal is the Wi-Fi signal, the sub-carrier selection module 122 may select the 3^{rd} to the 27^{th} sub-carriers and/or the 39^{th} to the 64^{th} sub-carriers of the Wi-Fi signal, so as to transmit the wireless signal or receive the echo of the wireless signal.

After receiving the echo through the transceiver 130, the data collection module 121 may obtain channel state information (CSI) from the echo. The detection module 123 may generate a detection result indicating whether an apnea event has occurred on the human subject according to the CSI. The output module 124 may output the detection result through the transceiver 130. For instance, the output module 124 may transmit the detection result to a terminal device (e.g., a smart phone) of a user. The user may interpret the detection result through the terminal device to determine whether the apnea event has occurred on the human subject during sleep.

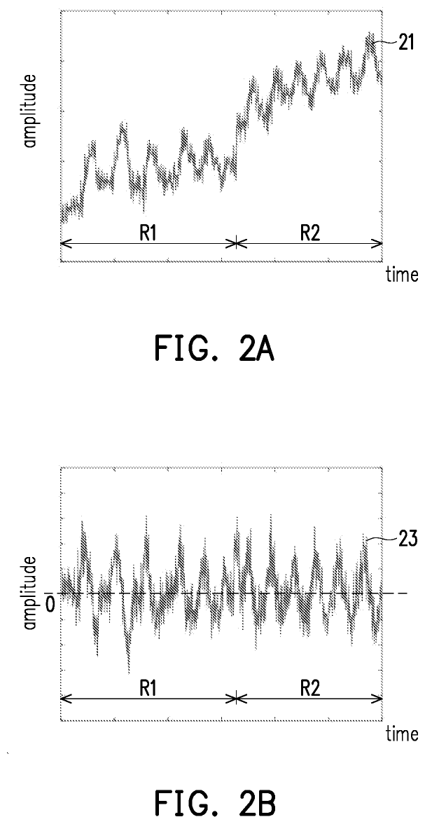

FIG. 2A is a schematic diagram illustrating the CSI according to an embodiment of the disclosure, wherein a curve 21 represents the CSI. It can be seen from FIG. 2A that there may exist an offset between the CSI during a time slot R1 and the CSI during a time slot R2. The offset may occur because of different sleep stages and may lead to misjudgment of the apnea event. In order to eliminate the offset, the detection module 123 may calculate a first-order differential signal corresponding to the CSI. FIG. 2B is a schematic diagram of a first-order differential signal according to an embodiment of the disclosure, wherein a curve 23 represents the first-order differential signal. It can be seen from FIG. 2B that there is no offset between the first-order differential signal during the time slot R1 and the first-order differential signal during the time slot R2.

In an embodiment, before calculating the first-order differential signal, the detection module 123 may generate a filtering signal corresponding to the CSI according to a filtering algorithm. FIG. 2C is a schematic diagram of a filtering signal according to an embodiment of the disclosure, wherein a curve 22 represents the filtering signal. After generating the filtering signal, the detection module 123 may differentiate the filtering signal to generate the first-order differential signal. The filtering algorithm may be associated with moving average filtering, high pass filtering, low pass filtering, or bandpass filtering, which should not be construed as a limitation in the disclosure.

In an embodiment, after the first-order differential signal is generated, the detection module 123 may correct an outlier in the first-order differential signal to generate a corrected first-order differential signal. FIG. 2D is a schematic diagram of a corrected first-order differential signal according to an embodiment of the disclosure, wherein the curve 23 represents the first-order differential signal that has not yet been corrected. The detection module 123 may generate an upper bound ub and a lower bound lb according to the median m of the first-order differential signal. For instance, the upper bound ub may be equal to the sum of the median m of the first-order differential signal and the standard deviation of the first-order differential signal, and the lower bound lb may be equal to the difference obtained by subtracting the standard deviation of the first-order differential signal from the median m of the first-order differential signal. Then, the detection module 123 may determine a data point larger than the upper bound ub or smaller than the lower bound lb in the first-order differential signal as an outlier. As shown in FIG. 2D, the detection module 123 may determine a data point PA smaller than the lower bound lb as the outlier.

The detection module 123 may correct the outlier in the first-order differential signal to generate a corrected first-order differential signal. In an embodiment, the detection module 123 may replace the outlier with the median m, so that the value of the corrected data point PA is the same as the median m. In an embodiment, the detection module 123 may perform an interpolation calculation on a data point PB earlier than the data point PA and a data point PC later than the data point PA in the first-order differential signal to correct the outlier. The corrected value of the data point PA may be equal to the interpolation calculation result of the data point PB and the data point PC.

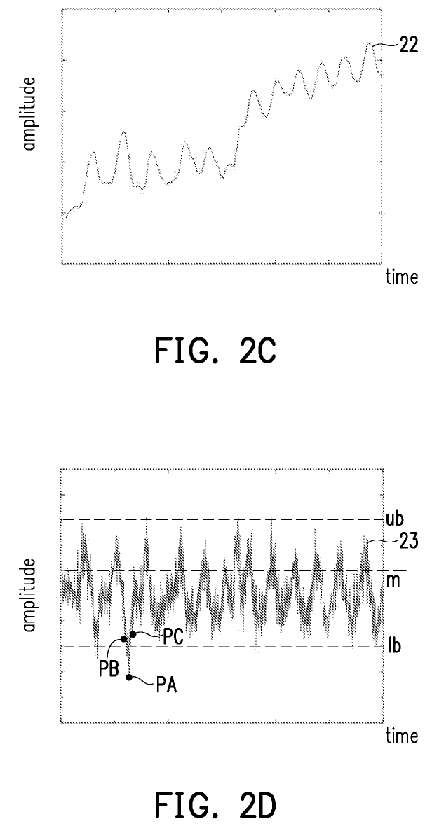

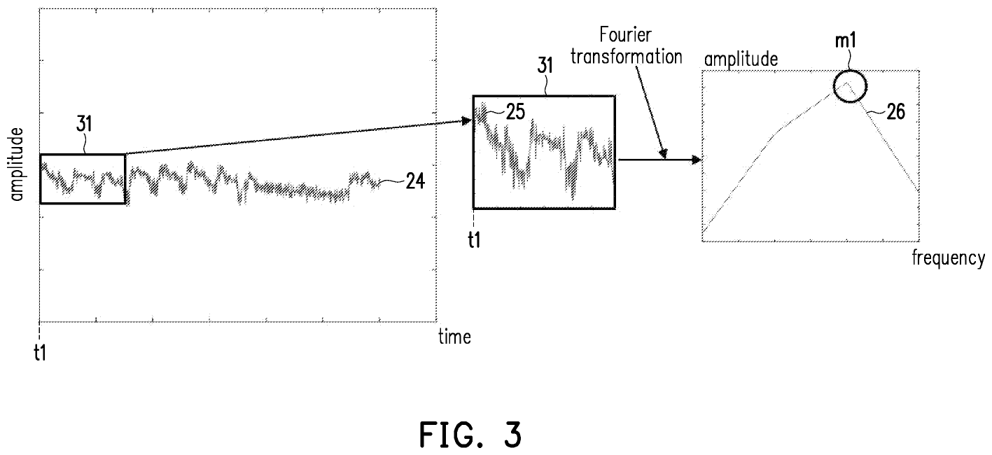

FIG. 3 is a schematic diagram of obtaining global maximum values of frequency responses according to an embodiment of the disclosure, wherein a curve 24 represents the first-order differential signal (or the corrected first-order differential signal). The detection module 123 may generate a plurality of frequency responses corresponding to the first-order differential signal, wherein the frequency responses may respectively correspond to a plurality of time points. For instance, the detection module 123 may apply a window function 31 to perform a Fourier transformation on a portion of the first-order differential signal 25 corresponding to a time point t1 to generate a frequency response corresponding to the time point t1, wherein the frequency response may be represented by a curve 26. The detection module 123 may further obtain a global maximum value ml corresponding to the frequency response of the time point t1. Through performing similar steps, the detection module 123 may obtain a plurality of global maximum values corresponding to the frequency responses. The detection module 123 may generate a global maximum value curve 27 corresponding to a reference time slot T1 according to the global maximum values and the time points corresponding to the global maximum values, as shown in FIG. 4.

FIG. 4 is a schematic diagram of determining an occurrence of an apnea event based on the global maximum values according to an embodiment of the disclosure, wherein the global maximum value curve 27 may include the global maximum values corresponding to the time points. For instance, the global maximum value curve 27 may include the global maximum value ml corresponding to the time point t1. The detection module 123 may detect the apnea event according to the global maximum value curve 27. Specifically, the detection module 123 may extract a reference time slot T2 from the reference time slot T1 according to a threshold TH. The reference time slot T2 corresponds to a subset of the global maximum values in the global maximum value curve 27, wherein each of the global maximum values in the subset is less than the threshold TH. As shown in FIG. 4, during the reference time slot T2, each of the global maximum values of the global maximum value curve 27 is less than the threshold TH.

After obtaining the reference time slot T2, the detection module 123 may determine whether the reference time slot T2 is greater than an apnea time slot. If the reference time slot T2 is greater than the apnea time slot, the detection module 123 may determine that the apnea event is detected. Accordingly, the detection module 123 may generate a detection result indicating the occurrence of the apnea event. The apnea time slot may be configured by the user of the electronic device 100. For instance, the user may configure the apnea time slot to ten seconds according to the definition of apnea in the medical community.

The threshold TH may be generated by the detection module 123. Specifically, the detection module 123 may arrange the global maximum values in the global maximum value curve 27 from small to large to generate a global maximum value sequence 28. Then, the detection module 123 may select an N^{th} global maximum value from the global maximum value sequence 28 as the threshold TH. The detection module 123 may obtain N according to equation (1), wherein T is the apnea time slot, T1 is the reference time slot, and K is the quantity of the global maximum values (i.e., the global maximum value sequence 28 contains K global maximum values).

$$N = ⌈ \frac{T}{T 1} \times K ⌉ \qquad ( 1 )$$

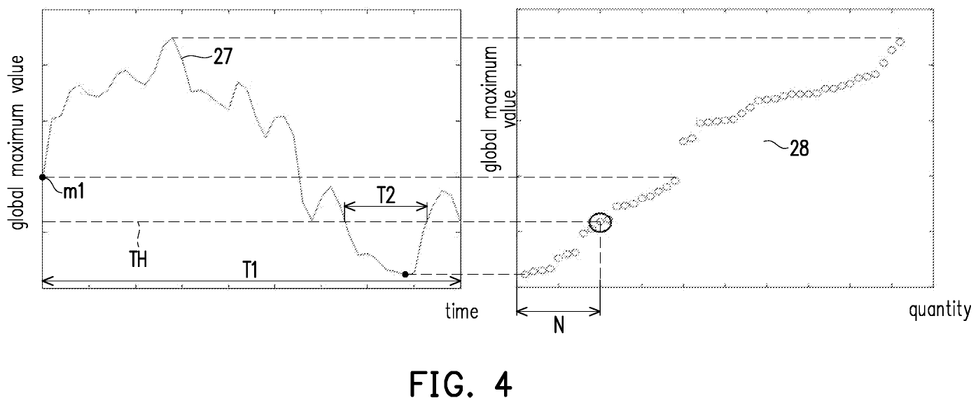

FIG. 5 is a schematic diagram of moving the head of a human subject 500 through an air cushion 300 according to an embodiment of the disclosure. The human subject may arrange the air cushion 300 at the bottom of a pillow 400. If the detection module 123 determines that the apnea event has occurred on the human subject, the air cushion control module 125 may inflate the air cushion 300 by configuring the air pump 200 through the transceiver 130. The air cushion 300 may raise the pillow 400, thereby raising the head 500 of the human subject to clear the airway of the human subject. Thereby, the occurrence of apnea event may be prevented.

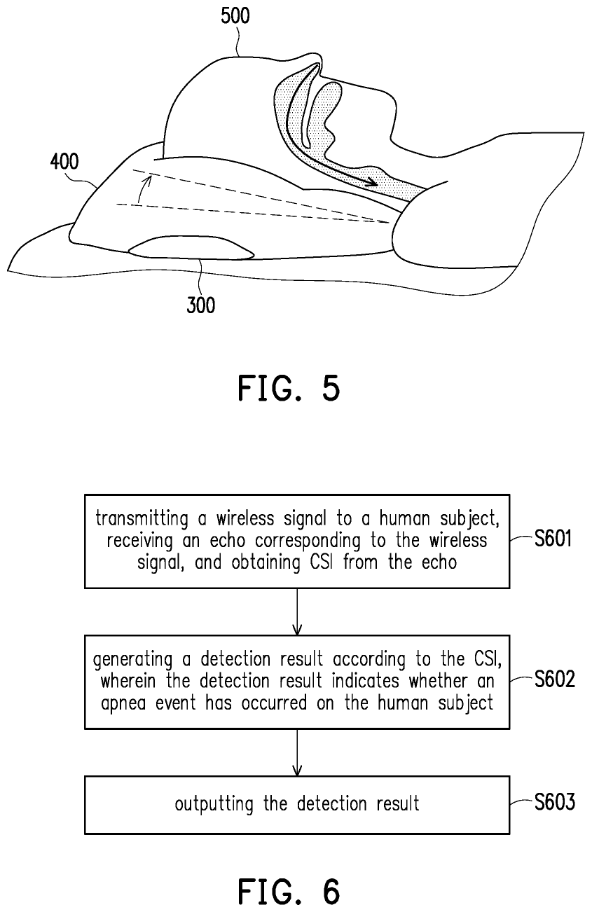

FIG. 6 is a flowchart of a method for detecting apnea according to an embodiment of the disclosure, where the method may be implemented by the electronic device 100 shown in FIG. 1. In step S601, a wireless signal is transmitted to a human subject, an echo corresponding to the wireless signal is received, and CSI is obtained from the echo. In step S602, a detection result is generated according to the CSI, wherein the detection result indicates whether an apnea event has occurred on the human subject. In step S603, the detection result is output.

To sum up, the electronic apparatus provided in one or more embodiments of the disclosure may be applied to detect the CSI without contacting the human subject and may still determine whether the apnea event has occurred on the human subject. There may exist an offset during the sleep time of the human subject (e.g., the offset that may occur because of different sleep stages and may affect the detection result. In response thereto, the CSI may be differentiated to eliminate the offset according to one or more embodiments of the disclosure. As provided in one or more embodiments of the disclosure, the frequency responses may serve to determine whether the apnea event occurs. If the apnea event occurs, the air cushion may be applied to raise the head of the human subject to open the human subject's respiratory tract in one or more embodiments of the disclosure, thereby preventing the occurrence of respiratory arrest events. As such, in one or more embodiments of the disclosure, whether the apnea event has occurred on the human subject can be monitored; what is more, the sleep quality of the human subject may also be improved.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An electronic device for detecting apnea, comprising:
a transceiver;
a storage medium, storing a plurality of modules; and
a processor, coupled to the storage medium and the transceiver and accessing and executing the modules, wherein the modules comprise:
a data collection module transmitting a wireless signal to a human subject through the transceiver, receiving an echo corresponding to the wireless signal through the transceiver, and obtaining channel state information from the echo;
a detection module, generating a detection result according to the channel state information, wherein the detection result indicates whether an apnea event has occurred on the human subject; and
an output module, outputting the detection result through the transceiver.

2. The electronic device according to claim 1, further comprising:
an air cushion; and
an air pump, connected to the air cushion and communicating with the transceiver, wherein the modules further comprise:
an air cushion control module, inflating the air cushion by configuring the air pump through the transceiver in response to the apnea event.

3. The electronic device according to claim 1, wherein the detection module is further configured to:
calculate a first-order differential signal corresponding to the channel state information; and
detect the apnea event according to the first-order differential signal.

4. The electronic device according to claim 3, wherein the detection module is further configured to:
generate a filtering signal corresponding to the channel state information according to a filtering algorithm; and
differentiate the filtering signal to generate the first-order differential signal.

5. The electronic device according to claim 4, wherein the filtering algorithm is associated with at least one of the following: moving average filtering, high pass filtering, low pass filtering, and bandpass filtering.

6. The electronic device according to claim 3, wherein the detection module is further configured to:
generate a plurality of frequency responses corresponding to the first-order differential signal, wherein the frequency responses respectively correspond to a plurality of time points;
obtain a plurality of global maximum values respectively corresponding to the frequency responses;
generate a global maximum value curve corresponding to a first reference time slot according to the global maximum values, wherein the first reference time slot comprises the time points; and
detect the apnea event according to the global maximum value curve.

7. The electronic device according to claim 6, wherein the detection module is further configured to:
extract a second reference time slot from the first reference time slot according to a threshold, wherein the second reference time slot corresponds to a subset of the global maximum values, and each of the global maximum values in the subset is less than the threshold; and
determine that the apnea event is detected in response to the second reference time slot being greater than an apnea time slot.

8. The electronic device according to claim 7, wherein the detection module is further configured to:
arrange the global maximum values from small to large to generate a global maximum value sequence; and
select an N^{th} global maximum value from the global maximum value sequence as the threshold, wherein N is a product of a ratio of the apnea time slot to the first reference time slot and the quantity of the global maximum values.

9. The electronic device according to claim 6, wherein the detection module is further configured to:
generate an upper bound and a lower bound according to a median of the first-order differential signal;
determine a data point larger than the upper bound or smaller than the lower bound in the first-order differential signal as an outlier; and
correct the outlier to generate a corrected first-order differential signal, and generate the frequency responses according to the corrected first-order differential signal.

10. The electronic device according to claim 9, wherein the detection module corrects the outlier according to one of following steps:
replacing the outlier with the median; and
performing an Interpolation calculation on a first data point earlier than the data point and a second data point later than the data point to correct the outlier, wherein the first data point and the second data point are included in the first-order differential signal.

11. A method of detecting apnea, comprising:
transmitting a wireless signal to a human subject, receiving an echo corresponding to the wireless signal, and obtaining channel state information from the echo;
generating a detection result according to the channel state information, wherein the detection result indicates whether an apnea event has occurred on the human subject; and
outputting the detection result.

12. The method according to claim 11, further comprising:
configuring an air pump to inflate an air cushion in response to the apnea event.

13. The method according to claim 11, wherein the step of generating the detection result according to the channel state information comprises:
calculating a first-order differential signal corresponding to the channel state information; and
detecting the apnea event according to the first-order differential signal.

14. The method according to claim 13, wherein the step of calculating the first-order differential signal corresponding to the channel state information comprises:
generating a filtering signal corresponding to the channel state information according to a filtering algorithm; and
differentiating the filtering signal to generate the first-order differential signal.

15. The method according to claim 14, wherein the filtering algorithm is associated with at least one of the following: moving average filtering, high pass filtering, low pass filtering, and bandpass filtering.

16. The method according to claim 13, wherein the step of detecting the apnea event according to the first-order differential signal comprises:
generating a plurality of frequency responses corresponding to the first-order differential signal, wherein the frequency responses respectively correspond to a plurality of time points;
obtaining a plurality of global maximum values respectively corresponding to the frequency responses;
generating a global maximum value curve corresponding to a first reference time slot according to the global maximum values, wherein the first reference time slot comprises the time points; and
detecting the apnea event according to the global maximum value curve.

17. The method according to claim 16, wherein the step of detecting the apnea event according to the global maximum value curve comprises:
extracting a second reference time slot from the first reference time slot according to a threshold, wherein the second reference time slot corresponds to a subset of the global maximum values, and each of the global maximum values in the subset is less than the threshold; and
determining that the apnea event is detected in response to the second reference time slot being greater than an apnea time slot.

18. The method according to claim 17, wherein the step of detecting the apnea event according to the global maximum value curve further comprises:
arranging the global maximum values from small to large to generate a global maximum value sequence; and
selecting an N^{th} global maximum value from the global maximum value sequence as the threshold, wherein N is a product of a ratio of the apnea time slot to the first reference time slot and the quantity of the global maximum values.

19. The method according to claim 16, wherein the step of generating the frequency responses corresponding to the first-order differential signal comprises:
generating an upper bound and a lower bound according to a median of the first-order differential signal;
determining a data point larger than the upper bound or smaller than the lower bound in the first-order differential signal as an outlier; and
correcting the outlier to generate a corrected first-order differential signal, and generating the frequency responses according to the corrected first-order differential signal.

20. The method according to claim 19, wherein the step of correcting the outlier to generate the corrected first-order differential signal comprises one of following steps:
replacing the outlier with the median; and
performing an interpolation calculation on a first data point earlier than the data point and a second data point later than the data point to update the outlier, wherein the first data point and the second data point are included in the first-order differential signal.
